(19) United States
(12) Patent Application Publication
(10) Pub. No.: US 2018/0064049 A1
(43) Pub. Date: Mar. 8, 2018

(54) SYSTEMS AND METHODS FOR DISPENSING POLLEN ONTO CROPS VIA UNMANNED VEHICLES

(71) Applicant: Wal-Mart Stores, Inc., Bentonville, AR (US)

(72) Inventors: Robert L. Cantrell, Herndon, VA (US); John P. Thompson, Bentonville, AR (US); David C. Winkle, Bella Vista, AR (US); Michael D. Atchley, Springdale, AR (US); Donald R. High, Noel, MO (US); Todd D. Mattingly, Bentonville, AR (US); Brian G. McHale, Greater Manchester (GB); John J. O'Brien, Farmington, AR (US); John F. Simon, Pembroke Pines, FL (US)

(21) Appl. No.: 15/697,004

(22) Filed: Sep. 6, 2017

Related U.S. Application Data
(60) Provisional application No. 62/384,906, filed on Sep. 8, 2016.

(51) Int. Cl. A01H 1/02; B64C 39/02; B64D 47/08; B64D 1/16; B64D 1/18

(52) U.S. Cl. CPC A01H 1/025; B64C 39/024; B05C 11/1026; B64D 1/16; B64D 1/18; B64D 47/08

(57) ABSTRACT

In some embodiments, methods and systems of pollinating crops in a crop-containing area include at least one unmanned vehicle having a receptacle including pollen, a pollen dispenser configured to dispense the pollen from the receptacle onto the crops, and a sensor configured to detect presence of the pollen dispensed from the at least one pollen dispenser on the crops and interpret the presence of the pollen dispensed from the at least one pollen dispenser on the crops as a verification that the pollen dispensed from the at least one pollen dispenser was successfully applied to the crops.

### CROSS-REFERENCE TO RELATED APPLICATION

This application claims the benefit of U.S. Provisional Application No. 62/384,906, filed Sep. 8, 2016, which is incorporated herein by reference in its entirety.

### TECHNICAL FIELD

This disclosure relates generally to pollinating crops, and in particular, to systems and methods for dispensing pollen onto crops using unmanned vehicles.

### BACKGROUND

Since most flowering crops rely on insects and/or animals for pollination, pollinators are very important to the maintenance of both wild and agricultural plant communities. In recent years, the amount of pollinators (e.g., ants, bees, beetles, butterflies, wasps, etc.) has been in steady decline, which leads to reduced fertility and biodiversity of the crops and reduced crop production. While there have been attempts to fertilize crops by pollinating the crops via crop dusting, blanket spraying of pollen onto the crops from an airplane flying above ground is non-targeted and a significant percentage of the pollen may not reach its intended target crops due to the speed of the moving airplane and intervening wind. In an attempt to ensure that a large percentage of crops in the crop-containing area are pollinated, the crop-duster planes often spray more pollen than would be necessary then if the pollination were targeted, making crop duster-based pollination more expensive. In addition, since crop-dusters merely spray the pollen with the hope of providing maximum pollen coverage, but do not provide any verification of which crops were successfully pollinated and which were not, a significant percentage of crops may remain non-pollinated despite the excessive amount of pollen sprayed by the crop-duster.

### BRIEF DESCRIPTION OF THE DRAWINGS

Disclosed herein are embodiments of systems, devices, and methods pertaining to pollinating crops via unmanned vehicles. This description includes drawings, wherein:

FIG. 1 is a diagram of a system for pollinating crops via unmanned aerial vehicles (UAVs) in accordance with some embodiments;

FIG. 2 comprises a block diagram of a UAV as configured in accordance with various embodiments of these teachings;

FIG. 3 is a functional block diagram of a computing device in accordance with some embodiments;

FIG. 4 is a flow diagram of a method of pollinating crops via UAVs in accordance with some embodiments; and

FIG. 5 is an enlarged fragmentary view of the pollen output device of FIG. 1 in accordance with some embodiments.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

The following description is not to be taken in a limiting sense, but is made merely for the purpose of describing the general principles of exemplary embodiments. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Generally, the systems, devices, and methods for pollinating crops in a crop-containing area include one or more unmanned vehicles having a receptacle including pollen, a pollen dispenser for dispensing the pollen from the receptacle on the crops, and a sensor configured to detect presence of the pollen dispensed from the pollen dispenser on the crops and interpret the presence of the pollen dispensed from the pollen dispenser on the crops as a verification that the pollen dispensed from the pollen dispenser was successfully applied to the crops.

In one embodiment, a system for pollinating crops in a crop-containing area includes at least one unmanned aerial vehicle having a receptacle including pollen, at least one pollen dispenser configured to dispense the pollen from the receptacle onto the crops when the at least one unmanned aerial vehicle is located above the crop-containing area, and at least one sensor configured to detect presence of the pollen dispensed from the at least one pollen dispenser on the crops and interpret the presence of the pollen dispensed from the at least one pollen dispenser on the crops as a verification that the pollen dispensed from the at least one pollen dispenser was successfully applied to the crops.

In another embodiment, a method of pollinating crops in a crop-containing area includes: providing at least one unmanned aerial vehicle having a receptacle including pollen, at least one pollen dispenser configured to dispense the pollen from the receptacle onto the crops when the at least one unmanned aerial vehicle is located above the crop-containing area, at least one sensor configured to detect presence of the pollen dispensed from the at least one pollen dispenser on the crops and interpret the presence of the pollen dispensed from the at least one pollen dispenser on the crops as a verification that the pollen dispensed from the at least one pollen dispenser was successfully applied to the crops; dispensing the pollen from the receptacle onto the crops via the at least one pollen dispenser; and detecting the presence of the pollen dispensed from the at least one pollen dispenser on the crops via the at least one sensor.

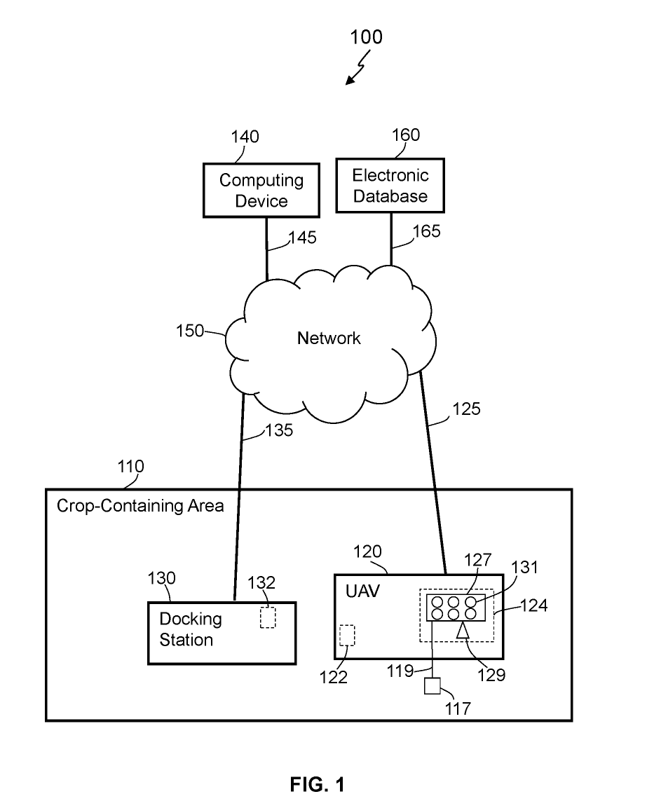

FIG. 1 illustrates an embodiment of a system 100 for dispensing pollen onto crops in a crop-containing area 110 and verifying that the crops were successfully pollinated with pollen. It will be understood that the details of this example are intended to serve in an illustrative capacity and are not necessarily intended to suggest any limitations in regards to the present teachings.

Generally, the exemplary system 100 of FIG. 1 includes a UAV 120 including a pollen output device 124 configured to dispense pollen from onto crops in a crop-containing area 110 and one or more sensors 122 configured to detect the presence of the pollen dispensed from the pollen output device 124 on the crops; a docking station 130 configured to permit the UAV 120 to land thereon and dock thereto to recharge; a processor-based computing device 140 in two-way communication with the UAV 120 (e.g., via communication channels 125 and 145) and/or docking station 130 (e.g., via communication channels 135 and 145) over the network 150; and an electronic database 160 in two-way communication with at least the computing device 140 (e.g., via communication channels 145 and 165) over the network 150. It is understood that more or fewer of such components may be included in different embodiments of the system 100.

As discussed above, while only one UAV 120 is shown in FIG. 1 for ease of illustration, it will be appreciated that in some embodiments, the computing device 140 may communicate with and/or provide flight route instructions and/or pollen dispensing instructions to two or more UAVs 120 simultaneously to guide the UAVs 120 along their predetermined routes to dispense pollen onto crops in the crop-containing area 110 and to detect the pollen dispensed by the UAVs 120 on the crops. Similarly, while only one docking station 130 is shown in FIG. 1, it will be appreciated that the system 100 may include two or more docking stations 130, where the UAVs 120 may dock in order to recharge and/or to refill the pollen output device 124 with pollen-containing solution 131 and/or to add or to other replace modular components of the UAV 120. In some aspects, the computing device 140 and the electronic database 160 may be implemented as separate physical devices as shown in FIG. 1 (which may be at one physical location or two separate physical locations), or may be implemented as a single device. In some embodiments, the electronic database 160 may be stored, for example, on non-volatile storage media (e.g., a hard drive, flash drive, or removable optical disk) internal or external to the computing device 140, or internal or external to computing devices distinct from the computing device 140. In some embodiments, the electronic database 160 is cloud-based.

Generally, the UAV 120 is configured to fly above ground through a space overlying the crop-containing area 110, to dispense pollen onto the crops when located above the crop-containing area 110, to detect the presence of pollen on the crops after dispensing the pollen, to land onto a docking station 130, and to dock onto the docking station 130 (e.g., for recharging), as described in more detail below. While the docking station 130 is shown in FIG. 1 as being located in the crop-containing area 110, it will be appreciated that one or more (or all) docking stations 130 may be positioned outside of the crop-containing area 110. The docking station 130 may be configured as an immobile station or a mobile (e.g., vehicle mounted) station. In some embodiments, the docking station 130 is optional to the system 100 and, in such embodiments, the UAV 120 is configured to take off from a deployment station (e.g., stand-alone or vehicle mounted) to initiate the dispensing of pollen onto crops in the crop-containing area 110, and to return to the deployment station without recharging after dispensing the pollen.

In some embodiments, the UAV 120 deployed in the exemplary system 100 does not require physical operation by a human operator and wirelessly communicates with, and is wholly or largely controlled by, the computing device 140. In particular, in some embodiments, the computing device 140 is configured to control directional movement and actions of the UAV 120 (e.g., flying, hovering, landing, taking off, moving while on the ground, dispensing pollen onto crops, detecting pollen on the crops, etc.) based on a variety of inputs. Generally, the UAV 120 of FIG. 1 is configured to move around the crop-containing area 110 (e.g., above ground or on the ground), dispense pollen from the pollen output device 124 onto the crops in the crop-containing area 110, and detect the pollen that was dispensed onto the crops in the crop-containing area 110 via one or more sensors 122.

While an unmanned aerial vehicle is generally described herein, in some embodiments, an aerial vehicle remotely controlled by a human may be utilized with the systems and methods described herein without departing from the spirit of the present disclosure. In some embodiments, the UAV 120 may be in the form of a multicopter, for example, a quadcopter, hexacopter, octocopter, or the like. In one aspect, the UAV 120 is an unmanned ground vehicle (UGV) that moves on the ground around the crop-containing area 110 under the guidance of the computing device 140 (or a human operator). In some embodiments, as described in more detail below, the UAV 120 includes a communication device (e.g., transceiver) configured to communicate with the computing device 140 while the UAV 120 is in flight and/or when the UAV 120 is docked at a docking station 130.

As described above, the exemplary UAV 120 shown in FIG. 1 includes at least one sensor 122 configured to detect the presence of pollen (e.g., pollen dispensed from the pollen output device 124) on the crops in the crop-containing area 110. In some embodiments, the sensor 122 of the UAV 120 is configured to interpret the presence of the pollen dispensed from the pollen output device 124 on the crops as a verification that the dispensed pollen was successfully applied to the crops. In some aspects, the sensor 122 is configured to merely detect the presence of the pollen dispensed by the pollen output device 124 on the crops and relay this detection data to another device (e.g., control circuit of the UAV 120, control circuit of the computing device 140, etc.) for interpreting this detection data as a verification that the pollen output device 124 successfully applied pollen onto the crops.

In some embodiments, the sensors 122 of the UAV 120 include a video camera configured to optically observe the presence of the pollen dispensed onto the crops by the pollen output device 124. In some embodiments, the video camera is a visible light camera, infrared camera, UV light camera, thermal camera, night-vision video camera, or the like cameras that are capable of providing a visual of the pollen as it appears on the crops (e.g., on leaves, flowers, fruits, or stalks). The sensors 122 of the UAV 120 may be configured to detect pollen on the crops during day or night pollination by the UAV 120. In some aspects, the video camera is configured as a radar-type scanner that identifies surface areas on the crops where pollen is detected as hot spots.

In some aspects, the sensors 122 of the UAV 120 are configured to detect the presence of pollen dispensed by the pollen output device 124 on the crops (e.g., flowers, fruits, leaves, stalks, etc.) and to capture the presence of the pollen on the crops as pollen detection data, which is then analyzed by the computing device 140 (or UAV 120) to determine the coverage of the crops with pollen. In some embodiments, after receiving pollen detection data indicating the detection of pollen dispensed from the UAV 120 on the crops in the crop-containing area 110 and determining that a high concentration of crops within a section of the crop-containing area 110 was not successfully pollinated, the computing device 140 is configured to send a control signal to the UAV 120 to instruct the UAV 120 to dispense additional pollen-containing solution 131 onto the crops in that section of the crop-containing area 110 via the pollen output device 124.

In some embodiments, as described in more detail below, the sensors 122 of the UAV 120 include one or more docking station-associated sensors including but not limited to: an optical sensor, a camera, an RFID scanner, a short range radio frequency transceiver, etc. Generally, the docking station-associated sensors of the UAV 120 are configured to detect and/or identify the docking station 130 based on guidance systems and/or identifiers of the docking station 130. For example, the docking station-associated sensor of the UAV 120 may be configured to capture identifying information of the docking station from one or more of a visual identifier, an optically readable code, a radio frequency identification (RFID) tag, an optical beacon, and a radio frequency beacon. In some embodiments, the sensors 122 of the UAV 120 may include other flight sensors such as optical sensors and radars for detecting obstacles (e.g., other UAVs 120) to avoid collisions with such obstacles.

In some embodiments, the pollen output device 124 of the UAV 120 is configured to dispense pollen from the UAV 120 onto the crops in the crop-containing area 110. In one aspect, the pollen output device 124 is configured to emit (e.g., via spray, aerosol, mist, or the like) a pollen-containing solution 131. In some embodiments, the pollen output device 124 includes a receptacle 127 (e.g., cartridge, canister, or the like) configured to contain the pollen-containing solution 131. In some aspects, the pollen containing solution 131 includes a binding agent that facilitates the adhesion of the pollen-containing solution 131 to the crops. Such a binding agent increases the efficacy of pollen application to the crops in that the pollen, upon coming into contact with a surface of a flower, is more likely to stay on the flower and not be blown off by the wind.

In one aspect, the pollen-containing solution contained in the receptacle 127 includes a detection facilitator agent (e.g., a dye, ink, or the like) configured to facilitate the detection of the presence of the pollen dispensed from the receptacle 127 on the crops by the sensor 122 of the UAV 120. In other words, after the pollen-containing solution 131 is dispensed onto the crops by the pollen output device 124, the detection facilitator agent, which is dispensed onto the crops together with the pollen-containing solution 131, emits a stimulus (e.g., visual, optical, reflective, chemical, heat/temperature, or the like.) that is detectable by one or more of the sensors 122 of the UAV 120 and, when detected, serves as a verification that the pollen dispensed by the pollen output device 124 of the UAV 120 was successfully applied to the crops.

The receptacle 127 of FIG. 1 is coupled to a pollen dispenser 129 (e.g., a nozzle, spout, valve, or the like) in fluid communication with the interior of the receptacle 127 and configured to dispense the pollen-containing solution 131 from the interior of the receptacle 127. As will be described in more detail below, in some aspects, the pollen dispenser 129 is coupled to a control signal-activated (e.g., piezoelectric) actuator configured to effectuate the release of pollen-containing solution 131 from the pollen dispenser 129 and/or a facilitator configured to facilitate the dispersion of the pollen as the pollen is being dispensed from the receptacle 127.

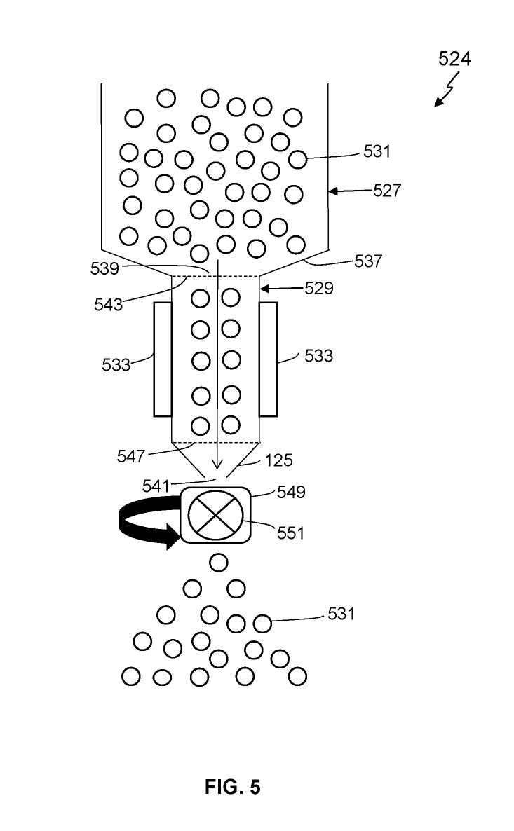

For example, as shown in FIG. 5, an exemplary pollen output device 524 includes a receptacle 527 including a solution 531 that includes pollen dispersed therein, a pollen dispenser 529 for dispensing the pollen-containing solution 531 from the receptacle 527 onto the crops, and a piezoelectric element 533 for activating the pollen dispenser 529.

The exemplary receptacle 527 of FIG. 5 includes a funnel 537 surrounding an opening 539 at a bottom of the receptacle 527 that is in fluid communication with the interior of the pollen dispenser 529, the bottom of which has an opening 541 through which the pollen-containing solution 531 is dispensed. While the funnel 537 facilitates the flow of the pollen-containing solution 531 in the downward direction indicated by the arrow toward the opening 541 of the pollen dispenser 529, in some embodiments, the bottom of the receptacle 527 is not configured in a funnel-like shape in some embodiments.

In the embodiment shown in FIG. 5, one valve 543 is configured to control the flow of the pollen-containing solution 531 through the opening 539 of the funnel 537 and into the interior of the pollen dispenser 529, and another valve 547 is configured to control the flow of the pollen-containing solution 531 through the opening 541 of the pollen dispenser 529. In some embodiments, the pollen dispenser 529 includes only valve 543 or only valve 547 and in some embodiments, the pollen dispenser 529 is configured without either valve 543 or valve 547 to dispense the pollen-containing solution 531 from the receptacle 527.

In some embodiments, the piezoelectric element 533 of FIG. 5, when activated, effectuates the release of droplets of pollen-containing solution 531 from the receptacle 527. The piezoelectric element 533 may be activated by a control signal generated internally to the UAV 120 (e.g., by a control circuit of the UAV 120), or remotely to the UAV 120 (e.g., by a control circuit of the computing device 140). In some aspects, after being activated by a control signal, the piezoelectric element 533 activates the pollen dispenser 529 (e.g., by opening the valves 543 and 547) to dispense the pollen-containing solution 531 from the pollen dispenser 529 through the opening 541 and onto the crops in the crop-containing area 110.

In the embodiment illustrated in FIG. 5, the pollen output device 524 includes a pollen dispersion facilitator 549 configured to disperse the pollen-containing solution 531 after the valve 547 is open and the pollen-containing solution flows through the opening 541 of the pollen dispenser 529. The pollen dispersion facilitator 549 includes one or more rotors 551 that rotate (e.g., in the direction indicated by the arrow in FIG. 5) to create turbulence around the opening 541 of the pollen dispenser 529. In some aspects, as the pollen-containing solution 531 flows through the opening 541, the turbulence created by the rotors 551 of the pollen dispersion facilitator 549 facilitates a mist-like dispersion of the pollen-containing solution 531 as shown in FIG. 5 and provides a wider coverage area for the pollen that is being dispensed by the pollen output device 524.

With reference to FIG. 1, the pollen output device 124 may be fully internal to the housing of the UAV 120, or may include one or more components that are external to the housing of the UAV 120. For example, in the embodiment illustrated in FIG. 1, the pollen output device 124 is operatively coupled to an element extending in part externally relative to the housing of the UAV 120 to disperse the pollen-containing solution 131 contained in the receptacle 127 onto the crops. Specifically, the exemplary UAV 120 of FIG. 1 includes an optional pollen applicator arm 119 extending downwardly from the housing of the UAV 120 and operatively coupled to a pollen applicator element 117 configured to apply the pollen-containing solution 131 onto the crops. In some aspects, the pollen applicator element 117 is a spreader, a brush, a pad, a cloth, a spray gun, or the like. The pollen applicator arm 119 may be configured so as to be in fluid communication with the receptacle 127 such that pollen-containing solution 131 can be delivered to the pollen applicator element 117 from the receptacle 127 via the pollen applicator arm 119. Examples of some suitable applicator arms are discussed in co-pending application entitled “SYSTEMS AND METHODS FOR POLLINATING CROPS VIA UNMANNED VEHICLES,” filed Sep. 8, 2016, which is incorporated by reference herein in its entirety.

In some embodiments, the pollen output device 124 is configured to be lowered from the housing of the UAV 120, for example, via an aerial crane. In some aspects, an aerial crane may be any device configured to move the pollen output device 124 between a retracted position that is closer to the housing of the UAV 120 and a deployed position that is further away from the housing of the UAV 120. For example, in some embodiments, an aerial crane may comprise one or more pulleys and extendable cables coupled to the pollen output device 124 via, for example, one or more of a hook, a latch, a clamp, a clip, a magnet, etc. In some embodiments, the aerial crane may be configured to unwind the cable to lower the pollen output device 124 toward the crops while the UAV 120 maintains a hover altitude (e.g. 5-10 feet above the crops). In some embodiments, the aerial crane may be configured to at least partially retract the cable into the housing of the aerial crane before the UAV 120 flies from one location in the crop-containing area 110 to another, or while the UAV 120 attempts to land onto or dock to a docking station 130. In some embodiments, the aerial crane may be controlled by a control circuit of the UAV 120. In some embodiments, the aerial crane may comprise a separate control circuit activated by the computing device 140 and/or a wireless transmitter of the docking 30.

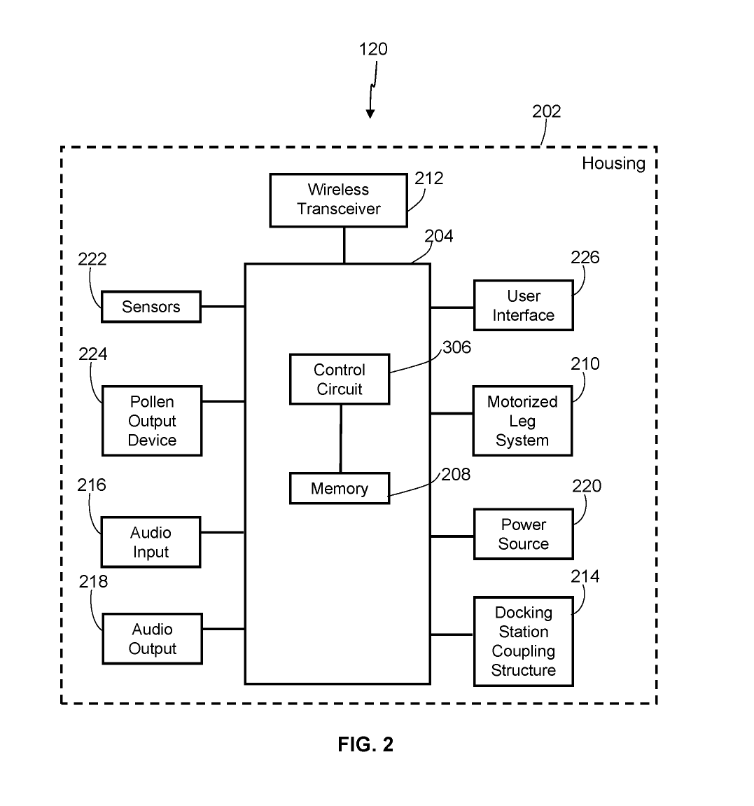

FIG. 2 presents a more detailed example of the structure of the UAV 120 of FIG. 1 according to some embodiments. The exemplary UAV 120 of FIG. 2 has a housing 202 that contains (partially or fully) or at least supports and carries a number of components. These components include a control unit 204 comprising a control circuit 206 that, like the control circuit 310 of the computing device 140, controls the general operations of the UAV 120. The control circuit 206 can comprise a fixed-purpose hard-wired platform or can comprise a partially or wholly programmable platform. These architectural options are well known and understood in the art and require no further description. The control circuit 206 is configured (e.g., by using corresponding programming stored in the memory 208 as will be well understood by those skilled in the art) to carry out one or more of the steps, actions, and/or functions described herein. The memory 208 may be integral to the control circuit 206 or can be physically discrete (in whole or in part) from the control circuit 206 as desired. This memory 208 can also be local with respect to the control circuit 206 (where, for example, both share a common circuit board, chassis, power supply, and/or housing) or can be partially or wholly remote with respect to the control circuit 206. The memory 208 can serve, for example, to non-transitorily store the computer instructions that, when executed by the control circuit 206, cause the control circuit 206 to behave as described herein. It is noted that not all components illustrated in FIG. 2 are included in all embodiments of the UAV 120. That is, some components may be optional depending on the implementation.

The control unit 204 of the UAV 120 of FIG. 2 includes a memory 208 coupled to the control circuit 206 for storing data (e.g., pollen detection data, instructions sent to the UAV 120 by the computing device 140, or the like). As discussed above, in some embodiments, the UAV 120 is not dependent on the electronic database 160 for storing pollen detection data, and on the computing device 140 for determining whether the crops targeted by the UAV 120 were successfully pollinated with the pollen dispensed by the UAV 120 and then sending a control signal to the UAV 120 indicating a suitable response output (e.g., dispensing of additional pollen-containing the pollen-dispensing solution 131) by the pollen output device 124. Instead, in some aspects, the memory 208 of the UAV 120 is configured to store pollen detection data and the control circuit 206 of the UAV 120 is programmed to analyze the pollen detection data captured by the sensors 122 of the UAV 120, determine whether the crops targeted by the UAV 120 were successfully pollinated with the pollen dispensed by the UAV 120 and then send a control signal to the pollen output device 124 indicating whether or not to dispense additional pollen onto the previously targeted crops. For example, in some embodiments, the control circuit 206 of the UAV 120 is programmed to determine (e.g., by analyzing the pollen detection data captured by the sensor 122) that the pollen dispensed onto the crops in a section of the crop-containing area 110 was not successfully applied onto the crops, for example, due to wind or rain interference, and to send a control signal to the pollen output device 124 to dispense additional pollen onto the crops in that section of the crop-containing area 110 accordingly.

In some embodiments, the control circuit 206 of the UAV 120 operably couples to a motorized leg system 210. This motorized leg system 210 functions as a locomotion system to permit the UAV 120 to land onto the docking station 130 and/or move while on the docking station 130. Various examples of motorized leg systems are known in the art. Further elaboration in these regards is not provided here for the sake of brevity save to note that the aforementioned control circuit 206 may be configured to control the various operating states of the motorized leg system 210 to thereby control when and how the motorized leg system 210 operates.

In the exemplary embodiment of FIG. 2, the control circuit 206 operably couples to at least one wireless transceiver 212 that operates according to any known wireless protocol. This wireless transceiver 212 can comprise, for example, a cellular-compatible, Wi-Fi-compatible, and/or Bluetooth-compatible transceiver that can wirelessly communicate with the computing device 140 via the network 150. So configured, the control circuit 206 of the UAV 120 can provide information to the computing device 140 (via the network 150), and can receive information and/or movement and/or pollen dispensing instructions from the computing device 140.

For example, the wireless transceiver 212 may be caused (e.g., by the control circuit 206) to transmit to the computing device 140, via the network 150, at least one signal indicating pollen detection data captured by a pollen-detecting sensor 122 of the UAV 120 while hovering over the crop-containing area 110. In some embodiments, the control circuit 206 receives instructions from the computing device 140 via the network 150 to dispense additional pollen via the pollen output device 124. In one aspect, the wireless transceiver 212 is caused (e.g., by the control circuit 206) to transmit an alert to the computing device 140, or to another computing device (e.g., hand-held device of a worker at the crop-containing area 110) indicating that one or more crops or crop sections in the crop-containing area 110 were not successfully pollinated by the pollen dispensed by the UAV 120. These teachings will accommodate using any of a wide variety of wireless technologies as desired and/or as may be appropriate in a given application setting. These teachings will also accommodate employing two or more different wireless transceivers 212, if desired.

The control circuit 206 also couples to one or more on-board sensors 222 of the UAV 120. These teachings will accommodate a wide variety of sensor technologies and form factors. As discussed above, the on-board sensors 222 of the UAV 120 can include sensors including but not limited to one or more sensors configured to detect the presence and/or location of pollen on crops (and on the ground adjacent to the crops) in the crop-containing area 110. Such sensors 222 can provide information (e.g., pollen detection data) that the control circuit 206 of the UAV 120 and/or the control circuit of the computing device 140 can analyze to determine whether the pollen dispensed from the pollen dispenser 129 of the UAV 120 was successfully applied to the crops. For example, in some embodiments, the UAV 120 includes an on-board sensor 222 in the form of a video camera configured to detect the presence of the pollen on the crops in the crop-containing area 110 and capture video-based pollen detection data that enables a visual confirmation of pollen presence.

In some embodiments, the sensors 222 of the UAV 120 are configured to detect objects and/or obstacles (e.g., other UAVs 120, docking stations 130, birds, animals, etc.) along the path of travel of the UAV 120. In some embodiments, using on-board sensors 222 (such as distance measurement units, e.g., laser or other optical-based distance measurement sensors), the UAV 120 may attempt to avoid obstacles, and if unable to avoid, the UAV 120 will stop until the obstacle is clear and/or notify the computing device 140 of such a condition.

By one optional approach, an audio input 216 (such as a microphone) and/or an audio output 218 (such as a speaker) can also operably couple to the control circuit 206 of the UAV 120. So configured, the control circuit 206 can provide for a variety of audible sounds to enable the UAV 120 to communicate with the docking station 130 or other UAVs 120. Such sounds can include any of a variety of tones and other non-verbal sounds.

In the embodiment of FIG. 2, the UAV 120 includes a rechargeable power source 220 such as one or more batteries. The power provided by the rechargeable power source 220 can be made available to whichever components of the UAV 120 require electrical energy. By one approach, the UAV 120 includes a plug or other electrically conductive interface that the control circuit 206 can utilize to automatically connect to an external source of electrical energy (e.g., charging dock 132 of the docking station 130) to recharge the rechargeable power source 220. By one approach, the UAV 120 may include one or more solar charging panels to prolong the flight time (or on-the-ground driving time) of the UAV 120.

These teachings will also accommodate optionally selectively and temporarily coupling the UAV 120 to the docking station 130. In such embodiments, the UAV 120 includes a docking station coupling structure 214. In one aspect, a docking station coupling structure 214 operably couples to the control circuit 206 to thereby permit the latter to control movement of the UAV 120 (e.g., via hovering and/or via the motorized leg system 210) towards a particular docking station 130 until the docking station coupling structure 214 can engage the docking station 130 to thereby temporarily physically couple the UAV 120 to the docking station 130. So coupled, the UAV 120 can recharge via a charging dock 132 of the docking station 130.

In some embodiments, the UAV 120 includes a pollen output device 224 coupled to the control circuit 206. Generally, the pollen output device 224 is configured to dispense pollen onto the crops in the crop-containing area 110. As discussed in more detail above with reference to the embodiment of FIG. 1, an exemplary pollen output device 224 may include a receptacle 127 including a pollen-containing solution 131 and a pollen dispenser 129 configured to dispense the pollen from the receptacle 127. In some embodiments, as described with reference to FIG. 5, the pollen output device 524 may include a piezoelectric element 533 configured to activate the pollen dispenser 529 and a pollen dispersion facilitator 549 configured to provide a wider coverage for the pollen being dispensed by the pollen dispenser 529.

In some embodiments, the UAV 120 includes a user interface 226 including for example, user inputs and/or user outputs or displays depending on the intended interaction with a user (e.g., operator of computing device 140) for purposes of, for example, manual control of the UAV 120, or diagnostics, or maintenance of the UAV 120. Some exemplary user inputs include but are not limited to input devices such as buttons, knobs, switches, touch sensitive surfaces, display screens, and the like. Example user outputs include lights, display screens, and the like. The user interface 226 may work together with or separate from any user interface implemented at an optional user interface unit (e.g., smart phone or tablet) usable by an operator to remotely access the UAV 120. For example, in some embodiments, the UAV 120 may be controlled by a user in direct proximity to the UAV 120 (e.g., a worker at the crop-containing area 110). This is due to the architecture of some embodiments where the computing device 140 outputs the control signals to the UAV 120. These controls signals can originate at any electronic device in communication with the computing device 140. For example, the movement signals sent to the UAV 120 may be movement instructions determined by the computing device 140 and/or initially transmitted by a device of a user to the computing device 140 and in turn transmitted from the computing device 140 to the UAV 120.

A docking station 130 of FIG. 1 is generally a device configured to permit at least one or more UAVs 120 to dock thereto. The docking station 130 may be configured as an immobile station (i.e., not intended to be movable) or as a mobile station (intended to be movable on its own, e.g., via guidance from the computing device 140, or movable by way of being mounted on or coupled to a moving vehicle), and may be located in the crop-containing area 110, or outside of the crop-containing area 110. For example, in some aspects, the docking station 130 may receive instructions from the computing device 140 over the network 150 to move into a position on a predetermined route of a UAV 120 over the crop-containing area 110.

In one aspect, the docking station 130 includes at least one charging dock 132 that enables at least one UAV 120 to connect thereto and charge. In some embodiments, a UAV 120 may couple to a charging dock 132 of a docking station 130 while being supported by at least one support surface of the docking station 130. In one aspect, a support surface of the docking station 130 may include one or more of a padded layer and a foam layer configured to reduce the force of impact associated with the landing of a UAV 120 onto the support surface of the docking station 130. In some embodiments, a docking station 130 may include lights and/or guidance inputs recognizable by the sensors of the UAV 120 when located in the vicinity of the docking station 130. In some embodiments, the docking station 130 may also include one or more coupling structures configured to permit the UAV 120 to detachably couple to the docking station 130 while being coupled to a charging dock 132 of the docking station 130.

In some embodiments, the docking station 130 is configured (e.g., by including a wireless transceiver) to send a signal over the network 150 to the computing device 140 to, for example, indicate if one or more charging docks 132 of the docking station 130 are available to accommodate one or more UAVs 120. In one aspect, the docking station 130 is configured to send a signal over the network 150 to the computing device 140 to indicate a number of charging docks 132 on the docking station 130 available for UAVs 120. The control circuit 310 of the computing device 140 is programmed to guide the UAV 120 to a docking station 130 moved into position along the predetermined route of the UAV 120 and having an available charging dock 132.

In some embodiments, a docking station 130 may include lights and/or guidance inputs recognizable by the sensors of the UAV 120 when located in the vicinity of the docking station 130. In some aspects, the docking station 130 and the UAV 120 are configured to communicate with one another via the network 150 (e.g., via their respective wireless transceivers) to facilitate the landing of the UAV 120 onto the docking station 130. In other aspects, the transceiver of the docking station 130 enables the docking station 130 to communicate, via the network 150, with other docking stations 130 positioned at the crop-containing area 110.

In some embodiments, the docking station 130 may also include one or more coupling structures configured to permit the UAV 120 to detachably couple to the docking station 130 while being coupled to a charging dock 132 of the docking station 130. In one aspect, the UAV 120 is configured to transmit signals to and receive signals from the computing device 140 over the network 150 only when docked at the docking station 130. For example, in some embodiments, after the pollen detection data captured by the sensors 122 of the UAV 120 is transmitted over the network 150 to the computing device 140 and the computing device 140 analyzes the pollen detection data to verify the presence of pollen dispensed by the UAV 120 on the crops, the UAV 120 is configured to receive a signal from the computing device 140 (containing instructions indicating whether the UAV 120 is to dispense additional pollen onto the crops) only when the UAV 120 is docked at the docking station 130. In other embodiments, the UAV 120 is configured to communicate with the computing device 140 and receive a signal from the computing device 140 (containing instructions indicating whether the UAV 120 is to dispense additional pollen onto the crops) while the UAV 120 is not docked at the docking station 130.

In some embodiments, the docking station 130 may be configured to not only recharge the UAV 120, but also to re-equip the pollen output device 124 of the UAV 120, and/or to add modular components to the pollen output device 124 of the UAV 120. For example, in some embodiments, the docking station 130 is configured to refill the receptacle 127 of the pollen output device 124 of the UAV 120 with pollen-containing solution 131 and/or replace the receptacle 127 with a new receptacle 127. In some embodiments, the docking station 130 is configured to provide for addition of new modular components to the pollen output device 124 of the UAV 120 (e.g., the above-discussed pollen applicator arm 119 may be coupled to the pollen output device 124 or uncoupled from the pollen output device 124 at the docking station 130.

In some embodiments, the docking station 130 may itself be equipped with a pollen output device akin to the pollen output device 124 of the UAV 120 to enable the docking station 130 to dispense pollen to the crops in the crop-containing area 110. As such, in some aspects of the system 100, the pollen can be dispensed not only by the UAV 120, but also by the docking station 130, thereby advantageously increasing the pollinating capabilities of the system 100.

In some embodiments, the docking station 130 is configured to provide for the addition of new modular components to the UAV 120 to enable the UAV 120 to better interact with the operating environment where the crop-containing area 110 is located. For example, in some aspects, the docking station 130 is configured to enable the coupling of various types of landing gear to the UAV 120 to optimize the ground interaction of the UAV 120 with the docking station 130 and/or to optimize the ability of the UAV 120 to land on the ground in the crop-containing area 110. In some embodiments, the docking station 130 is configured to enable the coupling of new modular components (e.g., rafts, pontoons, sails, or the like) to the UAV 120 to enable the UAV 120 to land on and/or move on wet surfaces and/or water. In some embodiments, the docking station 130 may be configured to enable modifications of the visual appearance of the UAV 120, for example, via coupling, to the exterior body of the UAV 120, one or more modular components (e.g., wings) designed to, for example, prolong the flight time of the UAV 120. It will be appreciated that the relative sizes and proportions of the docking station 130 and UAV 120 in FIG. 1 are not drawn to scale.

The computing device 140 of the exemplary system 100 of FIG. 1 may be a stationary or portable electronic device, for example, a desktop computer, a laptop computer, a tablet, a mobile phone, or any other electronic device. In some embodiments, the computing device 140 may comprise a control circuit, a central processing unit, a processor, a microprocessor, and the like, and may be one or more of a server, a computing system including more than one computing device, a retail computer system, a cloud-based computer system, and the like. Generally, the computing device 140 may be any processor-based device configured to communicate with the UAV 120, docking station 130, and electronic database 160 in order to guide the UAV 120 as it moves above ground or on the ground at the crop-containing area 110 and/or docks to a docking station 130 (e.g., to recharge) and/or deploys from the docking station 130 and/or dispenses pollen onto the crops in the crop-containing area 110.

The computing device 140 may include a processor configured to execute computer readable instructions stored on a computer readable storage memory. The computing device 140 may generally be configured to cause the UAVs 120 to: travel (e.g., fly, hover, or drive) around the crop-containing area 110, along a route determined by a control circuit of the computing device 140; detect the docking station 130 positioned along the route predetermined by the computing device 140; land on and/or dock to the docking station 130; undock from and/or lift off the docking station 130; dispense pollen onto the crops in the crop-containing area 110 via the pollen output device 124, and detect the presence of the pollen dispensed by the pollen output device 124 on the crops. In some embodiments, the electronic database 160 includes pollen detection data captured by the sensors 122 of the UAV 120 and transmitted to the electronic database 160 by the UAV 120 (e.g., via the computing device 140), and the computing device 140 is configured to analyze such pollen detection data and interpret the presence of the pollen dispensed from the pollen dispenser 129 on the crops as a verification that the pollen dispensed by the UAV 120 was successfully applied to the crops, and to instruct the UAV 120 to dispense additional pollen onto the crops, if the pollen verification data indicates that crops in one or more sections of the crop-containing area 110 were not successfully pollinated. In such embodiments, the pollen detection data is stored remotely to the UAV 120 and the determination of whether the pollen dispensed by the UAV 120 was successfully applied to the crops is made remotely to the UAV 120, namely, at the computing device 140, thereby reducing the data storage and processing power requirements of the UAV 120.

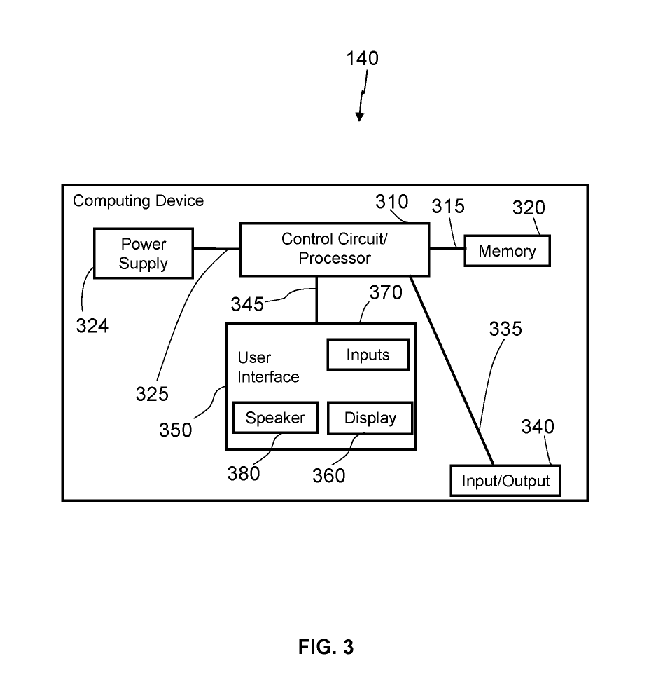

With reference to FIG. 3, a computing device 140 according to some embodiments configured for use with exemplary systems and methods described herein may include a control circuit 310 including a processor (e.g., a microprocessor or a microcontroller) electrically coupled via a connection 315 to a memory 320 and via a connection 325 to a power supply 330. The control circuit 310 can comprise a fixed-purpose hard-wired platform or can comprise a partially or wholly programmable platform, such as a microcontroller, an application specification integrated circuit, a field programmable gate array, and so on. These architectural options are well known and understood in the art and require no further description here.

The control circuit 310 can be configured (for example, by using corresponding programming stored in the memory 320 as will be well understood by those skilled in the art) to carry out one or more of the steps, actions, and/or functions described herein. In some embodiments, the memory 320 may be integral to the processor-based control circuit 310 or can be physically discrete (in whole or in part) from the control circuit 310 and is configured non-transitorily store the computer instructions that, when executed by the control circuit 310, cause the control circuit 310 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM)) as well as volatile memory (such as an erasable programmable read-only memory (EPROM))). Accordingly, the memory and/or the control circuit may be referred to as a non-transitory medium or non-transitory computer readable medium.

In some embodiments, the control circuit 310 of the computing device 140 is programmed to, in response to receipt (via the network 150) of pollen detection data (captured by the sensor 122 of the UAV 120) from the UAV 120, cause the computing device 140 to analyze such pollen detection data. In some aspects, the control circuit 310 of the computing device 140 is configured to transmit, over the network 150, the pollen detection data received from the UAV 120 to the electronic database 160, such that the electronic database 160 can be updated in real time to include up-to-date pollen detection information in the crop-containing area 110. In one aspect, the computing device 140 is configured to access, via the network 150, the pollen detection data stored on the electronic database 160 to determine whether the pollen dispensed by the UAV 120 onto the crops in the crop-containing area 110 is actually present on the crops.

In some embodiments, the control circuit 310 of the computing device 140 is programmed to generate a control signal to the UAV 120 based on a determination of whether the pollen detection data indicates that the targeted crops were successfully pollinated by the pollen dispensed by the UAV 120 or not. For example, such a control signal may instruct the UAV 120 to move toward a section of the crop-containing area 110 containing crops determined by the control circuit 310 of the computing device 140 as not having been successfully pollinated by the pollen dispensed by the UAV 120, and to dispense additional pollen over that section of the crop-containing area 110 in order to successfully pollinate the crops in that section. In some aspects, the control circuit 310 is programmed to cause the computing device 140 to transmit such control signal to the UAV 120 over the network 150.

The control circuit 310 of the computing device 140 is also electrically coupled via a connection 335 to an input/output 340 (e.g., wireless interface) that can receive wired or wireless signals from one or more UAVs 120. Also, the input/output 340 of the computing device 140 can send signals to the UAV 120, such as signals including instructions whether or not to dispense additional pollen onto the crops, or which docking station 130 the UAV 120 is to land on for recharging while hovering over the crop-containing area 110 along a route predetermined by the computing device 140.

In the embodiment shown in FIG. 3, the processor-based control circuit 310 of the computing device 140 is electrically coupled via a connection 345 to a user interface 350, which may include a visual display or display screen 360 (e.g., LED screen) and/or button input 370 that provide the user interface 350 with the ability to permit an operator of the computing device 140, to manually control the computing device 140 by inputting commands via touch-screen and/or button operation and/or voice commands to, for example, to send a signal to the UAV 120 in order to, for example: control directional movement of the UAV 120 while the UAV 120 is moving along a (flight or ground) route (over or on the crop-containing area 110) predetermined by the computing device 140; control movement of the UAV 120 while the UAV 120 is landing onto a docking station 130; control movement of the UAV 120 while the UAV is lifting off a docking station 130; control movement of the UAV 120 while the UAV 120 is in the process of dispensing pollen from the pollen output device 124; and/or control the movement of the UAV 120 while the UAV 120 attempts to detect whether the pollen dispensed by the pollen output device 124 was successfully applied onto the crops in the crop-containing area 110. Notably, the performance of such functions by the processor-based control circuit 310 of the computing device 140 is not dependent on actions of a human operator, and that the control circuit 310 may be programmed to perform such functions without being actively controlled by a human operator.

In some embodiments, the display screen 360 of the computing device 140 is configured to display various graphical interface-based menus, options, and/or alerts that may be transmitted from and/or to the computing device 140 in connection with various aspects of movement of the UAV 120 in the crop-containing area 110 as well as with various aspects of pollination of plants by the pollen output device 124 of the UAV 120 in response to the instructions received from the computing device 140. The inputs 370 of the computing device 140 may be configured to permit a human operator to navigate through the on-screen menus on the computing device 140 and make changes and/or updates to the routes of the UAV 120, pollen outputs of the pollen output device 124, and/or the locations of the docking stations 130. It will be appreciated that the display screen 360 may be configured as both a display screen and an input 370 (e.g., a touch-screen that permits an operator to press on the display screen 360 to enter text and/or execute commands.) In some embodiments, the inputs 370 of the user interface 350 of the computing device 140 may permit an operator to, for example, manually configure instructions to the UAV 120 for outputting pollen over a section of the crop-containing area 110 selected by the operator.

In some embodiments, the computing device 140 automatically generates a travel route for the UAV 120 from its deployment station to the crop-containing area 110, and to or from the docking station 130 while moving over or on the crop-containing area 110. In some embodiments, this route is based on a starting location of a UAV 120 (e.g., location of deployment station) and the intended destination of the UAV 120 (e.g., location of the crop-containing area 110, and/or location of docking stations 130 in or around the crop-containing area 110).

As discussed above, the electronic database 160 of FIG. 1 is configured to store electronic data including, but not limited to: pollen detection data captured by the sensors 122 of the UAV 120 after dispensing pollen onto the crops in the crop-containing area 110; data indicating location of the UAV 120 (e.g., GPS coordinates, etc.); data indicating level of pollen in the receptacle 127 of the pollen output device 124; data indicating locations within the crop-containing area 110 where additional pollen was dispensed by the UAV 120; route of the UAV 120 when moving from a deployment station to the crop-containing area 110, while flying over the crop-containing area 110, or when returning from the crop-containing area 110 to the deployment station; data indicating communication signals and/or messages sent between the computing device 140, UAV 120, electronic database 160, and/or docking station 130; data indicating location (e.g., GPS coordinates, etc.) of the docking station 130; and/or data indicating identity of one or more UAVs 120 docked at each docking station 130. As discussed above, in some embodiments, such electronic data is stored in the memory 208 of the UAV 120, such that the control circuit 206 of the UAV 120 accesses such electronic data from the memory 208 of the UAV 120 without having to access a remote electronic database over the network 150.

In some embodiments, location inputs are provided via the network 150 to the computing device 140 to enable the computing device 140 to determine the location of one or more of the UAVs 120 and/or one or more docking stations 130. For example, in some embodiments, the UAV 120 and/or docking station 130 may include a GPS tracking device that permits a GPS-based identification of the location of the UAV 120 and/or docking station 130 by the computing device 140 via the network 150. In one aspect, the computing device 140 is configured to track the location of the UAV 120 and docking station 130, and to determine, via the control circuit 310, an optimal route for the UAV 120 from its deployment station to the crop-containing area 110 and/or an optimal docking station 130 for the UAV 120 to dock to while traveling along its predetermined route. In some embodiments, the control circuit 310 of the computing device 140 is programmed to cause the computing device 140 to communicate such tracking and/or routing data to the electronic database 160 for storage and/or later retrieval.

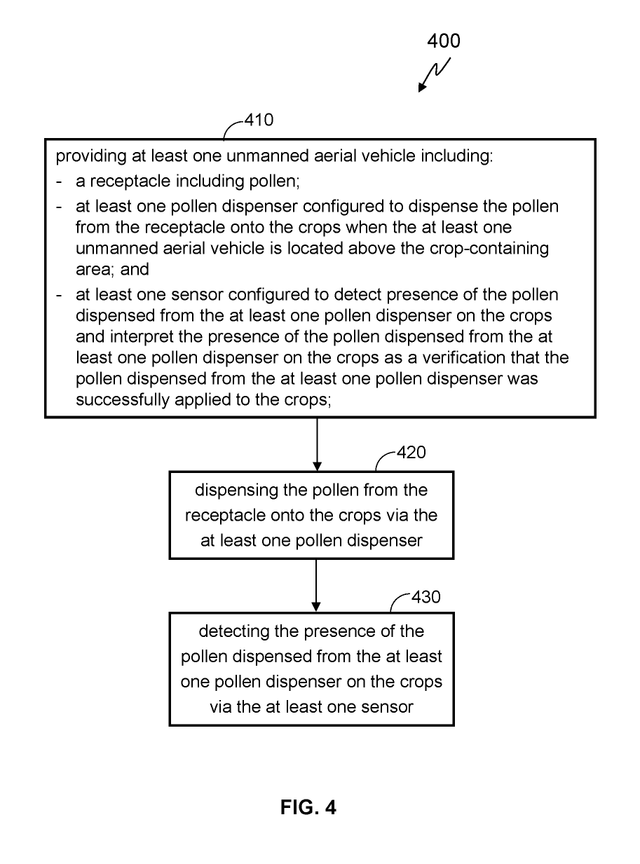

In view of the above description referring to FIGS. 1-3, and with reference to FIG. 4, a method 400 of pollinating crops in a crop-containing area 110 according to some embodiments will now be described. While the process 400 is discussed as it applies to dispensing pollen onto crops in a crop-containing area 110 and detecting the presence of the dispensed pollen on the crops and interpreting the presence of the dispensed pollen on the crops as a verification that the dispensed pollen was successfully applied to the crops via the exemplary system 100 shown in FIG. 1, it will be appreciated that the process 400 may be utilized in connection with any of the embodiments described herein.

The exemplary method 400 depicted in FIG. 4 includes providing one or more UAVs 120 including a receptacle 127 including a pollen-containing solution 131, one or more pollen dispensers 129 configured to dispense the pollen from the receptacle 127 onto the crops when the UAV 120 is located above the crop-containing area 110, and at least one sensor 122 configured to detect presence of the pollen dispensed from the pollen dispenser 129 on the crops and interpret the presence of the pollen dispensed from the pollen dispenser 129 on the crops as a verification that the dispensed pollen was successfully applied to the crops (step 410). The method further includes dispensing the pollen from the receptacle 127 onto the crops via one or more pollen dispensers 129 (step 420). As discussed above in more detail, in some embodiments, pollen is dispensed by the pollen output device 124 by way of being sprayed via the pollen dispenser 129 onto the crops and, in some embodiments, pollen is dispensed by the pollen output device 124 via a pollen applicator arm 119 extending from the UAV 120 and a pollen applicator element 117 (e.g., a spreader, a brush, a pad, a cloth, a spray gun, or the like.)

The method 400 of FIG. 4 further includes detecting the presence of the pollen dispensed from the pollen dispenser 129 on the crops via one or more sensors 122 of the UAV 120 (step 430). As discussed above in more detail, in some embodiments, the sensors 122 of the UAV 120 include a camera capable of capturing pollen detection data that provides an optical-based, chemical-based, or heat/temperature-based indication of the pollen as it appears on the crops (e.g., on leaves, flowers, fruits, or stalks). This pollen detection data is then analyzed (e.g., by the computing device 140 or by the UAV 120) in order to determine how successfully the pollen was applied to the crops (e.g., calculate a percentage of the crops in the crop-containing area 110 successfully covered by the pollen dispensed by the UAV 120). In some embodiments, after collection, by the UAV 120, of pollen detection data indicating the detection of pollen dispensed from the UAV 120 on the crops in the crop-containing area 110, and after a determination by the computing device 140 as to whether additional pollen needs to be dispensed onto the crops in the crop-containing area 110, the method further includes sending a control signal to the UAV 120 over the network 150 from the computing device 140 to instruct the UAV 120 to dispense additional pollen-containing solution 131 onto the crops in a section of the crop-containing area 110 determined to have crops that were not successfully pollinated when the pollen was initially dispensed by the pollen output device 124 of the UAV 120.

The systems and methods described herein advantageously provide for semi-automated or fully automated targeted dispensing of pollen on crops in in crop-containing areas via unmanned vehicles and detecting whether the dispensed pollen was successfully applied onto the crops intended to be pollinated. As such, the present systems and methods significantly reduce the amount of pollen that needs to be dispensed and significantly reduce the resources needed to determine whether the pollen was successfully applied onto the crops, thereby, thereby advantageously providing an efficient, self-sufficient, and cost-effective pollination system.

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. A system for pollinating crops in a crop-containing area, the system comprising:
at least one unmanned aerial vehicle including:
a receptacle including pollen;
at least one pollen dispenser configured to dispense the pollen from the receptacle onto the crops when the at least one unmanned aerial vehicle is located above the crop-containing area; and
at least one sensor configured to detect presence of the pollen dispensed from the at least one pollen dispenser on the crops and interpret the presence of the pollen dispensed from the at least one pollen dispenser on the crops as a verification that the pollen dispensed from the at least one pollen dispenser was successfully applied to the crops.

2. The system of claim 1, wherein the at least one sensor of the at least one unmanned aerial vehicle includes a video camera configured to optically observe the presence of the pollen dispensed from the at least one pollen dispenser on the crops.

3. The system of claim 1, wherein the receptacle further includes a detection facilitator agent configured to enable the at least one sensor to detect the presence of the pollen dispensed from the at least one pollen dispenser on the crops, the pollen dispenser being configured to dispense the pollen on the crops from the receptacle together with the detection facilitator agent.

4. The system of claim 1, wherein the receptacle includes the pollen dispersed in a solution, the pollen dispenser further comprising a piezoelectric element configured to effectuate release of pollen-containing droplets from the receptacle in response to activation of the piezoelectric element.

5. The system of claim 1, wherein the receptacle is a funnel configured to retain the pollen, and wherein the at least one pollen dispenser includes a valve configured to control flow of the pollen through the funnel, and at least one rotor configured to disperse the pollen when the valve is open to permit the flow of the pollen through the funnel.

6. The system of claim 1, wherein the at least one pollen dispenser includes at least one pollen applicator arm extending downward from the at least one unmanned aerial vehicle and operatively coupled to at least one of: a spreader, a brush, a pad, a cloth, and a spray gun.

7. The system of claim 6, wherein the pollen dispenser is configured to move the at least one pollen applicator arm in a downward direction toward the crops while the at least one unmanned aerial vehicle hovers over the crops.

8. The system of claim 1, further comprising at least one docking station positioned proximate the crop-containing area and configured to accommodate the at least one unmanned aerial vehicle; and
a computing device including a processor-based control circuit and configured to communicate with the at least one unmanned aerial vehicle and the at least one docking station via a network.

9. The system of claim 8, wherein the at least one unmanned aerial vehicle is configured to send a first signal to the computing device via the network, the first signal including pollen detection data captured by the at least one sensor of the at least one unmanned aerial vehicle upon detection, by the at least one sensor, of the presence of the pollen dispensed from the at least one pollen dispenser on the crops, and wherein the control circuit of the computing device is programmed to control at least one of movement, altitude, and dispensing of pollen by the at least one unmanned aerial vehicle based on the first signal.

10. The system of claim 9, further comprising an electronic database in communication with at least one of the computing device and the at least one unmanned aerial vehicle, the electronic database configured to store the pollen detection data received by the computing device from the at least one unmanned aerial vehicle.

11. A method of pollinating crops in a crop-containing area, the method comprising:
providing at least one unmanned aerial vehicle including:
a receptacle including pollen;
at least one pollen dispenser configured to dispense the pollen from the receptacle onto the crops when the at least one unmanned aerial vehicle is located above the crop-containing area; and
at least one sensor configured to detect presence of the pollen dispensed from the at least one pollen dispenser on the crops and interpret the presence of the pollen dispensed from the at least one pollen dispenser on the crops as a verification that the pollen dispensed from the at least one pollen dispenser was successfully applied to the crops;
dispensing the pollen from the receptacle onto the crops via the at least one pollen dispenser; and
detecting the presence of the pollen dispensed from the at least one pollen dispenser on the crops via the at least one sensor.

12. The method of claim 11, wherein the providing step further comprising providing the at least one sensor in a form of a video camera configured to optically observe the presence of the pollen dispensed from the at least one pollen dispenser on the crops.

13. The method of claim 11, further comprising:
providing a detection facilitator agent in the receptacle, the detection facilitator agent configured to enable the at least one sensor to detect the presence of the pollen dispensed from the at least one pollen dispenser on the crops; and
dispensing, via the pollen dispenser, the pollen on the crops from the receptacle together with the detection facilitator agent.

14. The method of claim 11, further comprising:
providing the pollen dispersed in a solution in the receptacle; and
providing the pollen dispenser with a piezoelectric element configured to effectuate release of pollen-containing droplets from the receptacle in response to activation of the piezoelectric element.

15. The method of claim 11, further comprising:
providing the receptacle in a form of a funnel configured to retail the pollen; and
providing the pollen dispenser with a valve configured to control flow of the pollen through the funnel and with a at least one rotor configured to disperse the pollen when the valve is open to permit the flow of the pollen through the funnel.

16. The method of claim 11, wherein the providing step further comprises providing the at least one unmanned aerial vehicle with at least one pollen applicator arm extending downward from the at least one unmanned aerial vehicle and operatively coupled to at least one of: a spreader, a brush, a pad, a cloth, and a spray gun.

17. The method of claim 16, further comprising moving, via the pollen dispenser, the at least one pollen applicator arm in a downward direction toward the crops while the at least one unmanned aerial vehicle hovers over the crops.

18. The method of claim 11, further comprising:
providing at least one docking station positioned proximate the crop-containing area and configured to accommodate the at least one unmanned aerial vehicle; and
providing a computing device including a processor-based control circuit and configured to communicate with the at least one unmanned aerial vehicle and the at least one docking station via a network.

19. The method of claim 18, further comprising:
sending, from the at least one unmanned aerial vehicle to the computing device via the network, a first signal including pollen detection data captured by the at least one sensor of the at least one unmanned aerial vehicle upon detection, by the at least one sensor, of the presence of the pollen dispensed from the at least one pollen dispenser on the crops; and
controlling, via the control circuit of the computing device, at least one of movement, altitude, and dispensing of pollen by the at least one unmanned aerial vehicle based on the first signal.

20. The method of claim 19, further comprising providing an electronic database in communication with at least one of the computing device and the at least one unmanned aerial vehicle, the electronic database configured to store the pollen detection data received by the computing device from the at least one unmanned aerial vehicle.
